(19) United States
(12) Patent Application Publication
(10) Pub. No.: US 2019/0125178 A1
(43) Pub. Date: May 2, 2019

(54) OPHTHALMIC IMAGING DEVICE

(71) Applicant: NIDEK CO., LTD., Gamagori-shi (JP)

(72) Inventor: Keiji MURATA, Toyama-shi (JP)

(73) Assignee: NIDEK CO., LTD., Gamagori-shi (JP)

(21) Appl. No.: 16/173,774

(22) Filed: Oct. 29, 2018

(30) Foreign Application Priority Data
Oct. 31, 2017 (JP) 2017-211370

(51) Int. Cl. A61B 3/00; A61B 3/10; A61B 3/117; A61B 3/12; A61B 3/14; A61B 5/00

(52) U.S. Cl. CPC A61B 3/0025; A61B 3/102; A61B 3/1025; A61B 5/0066; A61B 3/1225; A61B 3/14; A61B 3/117

(57) ABSTRACT

An ophthalmic imaging device includes a first optical system, a second optical system, and a wavelength splitting member. The first optical system irradiates light whose center wavelength is a first wavelength region being an infrared region. The first optical system includes an optical coherence tomography unit and an imaging element. The second optical system irradiates light whose center wavelength is in a second wavelength region. A wavelength of the second wavelength region is shorter than a wavelength of the first wavelength region. The second optical system includes a fixation light source and a short wavelength optical system. The wavelength splitting member is disposed on a common optical path of the measuring light, an anterior ocular segment photographing light, a fixation light, and a short wavelength light. The wavelength splitting member divides the common optical path into a first optical path and a second optical path.

### CROSS REFERENCE TO RELATED APPLICATION

This application is based upon and claims the benefit of priority of Japanese Patent Application No. 2017-211370 filed on Oct. 31, 2017, the contents of which are incorporated herein by reference in its entirety.

### BACKGROUND

The present disclosure relates to an ophthalmic imaging device.

Conventionally, an ophthalmic imaging device is known that includes an optical coherence tomography (OCT) unit, an imaging element, a fixation light source, and an optical system. The imaging element captures an image of an anterior ocular segment of a subject's eye. The fixation light source projects a vision fixation target onto the subject's eye. The optical system performs at least one of capturing an image of the ocular fundus of the subject's eye and irradiating treatment light onto the subject's eye. For example, a known ophthalmic inspection device includes an OCT optical system, which is an example of the optical coherence tomography unit, a photodetection element of an anterior ocular segment observation optical system, a fixation target display body, which is an example of the fixation light source, and a fundus camera optical system. The photodetection element of the anterior ocular segment observation optical system captures an image of the anterior ocular segment of the subject's eye. An optical path on which the subject's eye is disposed is divided into an optical path on which the OCT optical system and the anterior ocular segment observation optical system are disposed and an optical path on which the fixation light source and the fundus camera optical system are disposed, by using a dichroic mirror, which is an example of a wavelength splitting member.

### SUMMARY

In the optical coherence tomography unit, various OCT light sources having different center wavelengths may be used. For example, the center wavelength of the OCT light source used in SD-OCT and the center wavelength of the OCT light source used in SS-OCT may be different. When manufacturing a plurality of types of ophthalmic imaging devices having OCT light sources with mutually different center wavelengths, there is a need to perform adjustment in accordance with the OCT light source. The adjustment includes replacement, position change, and the like of an optical member, for example. Further, in order to reduce the manhours for the adjustment, there is a case in which a wavelength splitting member that can be used commonly for the plurality of types of the OCT light sources is used. In this case, it may be necessary to divide the optical path of a plurality of optical systems having similar wavelengths, by using the wavelength splitting member. Thus, there is a need for the wavelength splitting member to have a high performance.

Embodiments of the broad principles derived herein provide an ophthalmic imaging device in which a plurality of types of OCT light sources having different center wavelengths can be selectively installed in an easy manner.

Embodiments provide an ophthalmic imaging device that includes a first optical system, a second optical system, and a wavelength splitting member. The first optical system irradiates light whose center wavelength is a first wavelength region. The first wavelength region is an infrared region. The first optical system includes an optical coherence tomography unit and an imaging element. The optical coherence tomography unit acquires an OCT signal of a tissue of a subject's eye by dividing OCT light emitted from an OCT light source into measuring light and reference light and receiving interference light of the reference light and reflected light of the measuring light reflected by the tissue. The imaging element captures an image of an anterior ocular segment of the subject's eye by receiving anterior ocular segment photographing light that is infrared light reflected by the anterior ocular segment of the subject's eye. The second optical system irradiates light whose center wavelength is in a second wavelength region. A wavelength of the second wavelength region is shorter than a wavelength of the first wavelength region. The second optical system includes a fixation light source and a short wavelength optical system. The fixation light source projects a fixation target onto the subject's eye by emitting fixation light that is visible light. The short wavelength optical system performs at least one of capturing an image of an ocular fundus of the subject's eye and irradiating treatment light onto the subject's eye by using short wavelength light. The short wavelength light has a wavelength that is shorter than a wavelength of the measuring light and shorter than a wavelength of the anterior ocular segment photographing light. The wavelength splitting member is disposed on a common optical path of the measuring light, the anterior ocular segment photographing light, the fixation light, and the short wavelength light. The wavelength splitting member divides the common optical path into a first optical path and a second optical path by splitting light that passes through a mirror surface of the wavelength splitting member and light that is reflected by the mirror surface. The first optical path is an optical path through which the measuring light and the anterior ocular segment photographing light pass. The second optical path is an optical path through which the fixation light and the short wavelength light pass. An angle formed between the mirror surface and an optical axis of the common optical path is more than 45 degrees and less than 90 degrees.

Embodiments further provide an ophthalmic imaging device that includes a first optical system, a second optical system, and a wavelength splitting member. The first optical system includes an optical coherence tomography unit. The optical coherence tomography unit acquires an OCT signal of a tissue of a subject's eye by dividing OCT light emitted from an OCT light source into measuring light and reference light and receiving interference light of the reference light and reflected light of the measuring light reflected by the tissue. The second optical system irradiates light in a second wavelength region onto the subject's eye. A wavelength of the second wavelength region is shorter than a wavelength of a first wavelength region including a wavelength region of the OCT light. The first wavelength region includes a wavelength of an OCT light source used in SD-OCT and a wavelength of an OCT light source used in SS-OCT. The wavelength splitting member is disposed on a common optical path of light irradiated from the first optical system and light irradiated from the second optical system. The wavelength splitting member divides the common optical path into a first optical path and a second optical path by splitting light that passes through a mirror surface of the wavelength splitting member and light that is reflected by the mirror surface. The first optical path is a path through which light in the first wavelength region passes. The second optical path is a path through which the light in the second wavelength region passes.

### BRIEF DESCRIPTION OF THE DRAWINGS

FIG. 1 is a diagram showing a general configuration of an ophthalmic imaging device 1.

FIG. 2 is a diagram illustrating an optical system disposed on a measuring light optical path 112.

### DETAILED DESCRIPTION

### Overview

An aspect of an ophthalmic imaging device exemplified in the present disclosure includes a first optical system, which irradiates light whose center wavelength is a first wavelength region that is an infrared region, and a second optical system, which irradiates light whose center wavelength is in a second wavelength region whose wavelength is shorter than a wavelength of the first wavelength region. The first optical system includes an optical coherence tomography unit and an imaging element. The optical coherence tomography unit acquires an OCT signal of a tissue of a subject's eye by dividing OCT light emitted from an OCT light source into measuring light and reference light and receiving interference light of the reference light and reflected light of the measuring light reflected by the tissue. The imaging element captures an image of an anterior ocular segment of the subject's eye by receiving anterior ocular segment photographing light that is infrared light reflected by the anterior ocular segment of the subject's eye. The second optical system includes a fixation light source and a short wavelength optical system. The fixation light source projects a fixation target onto the subject's eye by emitting fixation light that is visible light. The short wavelength optical system performs at least one of capturing an image of an ocular fundus of the subject's eye and irradiating treatment light onto the subject's eye by using short wavelength light. The short wavelength light has a wavelength that is shorter than a wavelength of the measuring light and shorter than a wavelength of the anterior ocular segment photographing light. The ophthalmic imaging device further includes a wavelength splitting member. The wavelength splitting member is disposed on a common optical path of the measuring light, the anterior ocular segment photographing light, the fixation light, and the short wavelength light. The wavelength splitting member divides the common optical path into a first optical path and a second optical path by splitting light that passes through a mirror surface of the wavelength splitting member and light that is reflected by the mirror surface. The first optical path is an optical path through which the measuring light and the anterior ocular segment photographing light pass. The second optical path is an optical path through which the fixation light and the short wavelength light pass. An angle formed between the mirror surface and an optical axis of the common optical path is more than 45 degrees and less than 90 degrees.

In this case, the wavelength splitting member can accurately divide the common optical path into the first optical path and the second optical path. Thus, a necessity to perform adjustment along the optical path of the short wavelength light and the optical path of the fixation light in accordance with the type of the OCT light source can be reduced. Further, it is sufficient that the wavelength splitting member be configured to be capable of dividing the common optical path into the optical path of the measuring light and the anterior ocular segment photographing light and the optical path of the short wavelength light and the fixation light. Thus, the wavelength splitting member need not necessarily have bandpass characteristics that only allow light of a wavelength of a particular range to pass through or be reflected, for example. Further, by making the angle formed between the mirror surface of the wavelength splitting member and the optical axis of the common optical path to be more than 45 degrees and less than 90 degrees, the wavelength splitting member is not necessarily required to have high accuracy (mirror thickness, coating, and the like, for example). As a result, a plurality of types of the OCT light sources having mutually different center wavelengths can easily be selectively installed in the ophthalmic photographing device.

Preferably, the angle formed between the mirror surface of the wavelength splitting member and the optical axis of the common optical path may be more than 45 degrees and less than 80 degrees. More preferably, the angle formed between the mirror surface of the wavelength splitting member and the optical axis of the common optical path may be more than 45 degrees and less than 70 degrees.

An achromatic lens, which corrects chromatic aberration of the measuring light, may be included among one or a plurality of lenses disposed on an optical path of the measuring light. In this case, by correcting the chromatic aberration of the measuring light using the achromatic lens, the optical coherence tomography unit can acquire the OCT signal in which the influence of the aberration is suppressed. In this way, on the basis of the OCT signal, clearer data (tomographic image data, for example) can be generated.

For example, both the correction of chromatic aberration at wavelengths in the vicinity of W1 nm (wavelengths from 800 nm to 900 nm, for example) and the correction of chromatic aberration at wavelengths in the vicinity of W2 nm W1 nm) (wavelengths from 1000 nm to 1100 nm, for example) may be possible by the achromatic lens. Further, for example, the correction of chromatic aberration from a wavelength in the vicinity of W1 nm to a wavelength in the vicinity of W2 nm (wavelengths from 800 nm to 1100 nm, for example) may be possible by the achromatic lens.

The achromatic lens may include at least one positive lens and at least one negative lens. The achromatic lens may be a cemented lens in which the at least one positive lens and the at least one negative lens are joined together.

Preferably, the achromatic lens may include one positive lens and one negative lens. In this case, in comparison to a case in which there is a plurality of at least one of the positive lens and the negative lens, the number of reflection surfaces of the lenses becomes small. Thus, the possibility of distortion of the OCT signal by the reflection of the light by the lens can be reduced.

The ophthalmic imaging device need not necessarily include the achromatic lens.

A coating, which suppresses reflection of a plurality of types of the OCT light having different center wavelengths, may be applied to at least one lens among one or a plurality of lenses disposed on an optical path of the measuring light. In this case, even when the OCT light source is replaced, the reflection of the measuring light by the lens can be suppressed. Thus, it is possible to use a greater number of shared components.

The coating may suppress both the reflection of light of a wavelength in the vicinity of 850 nm and the reflection of light of a wavelength in the vicinity of 1050 nm, for example.

When the OCT light source is replaced, at least one of one or a plurality of the lenses disposed on the optical path of the measuring light may be replaced by a lens that is appropriate for the wavelength of the OCT light source.

The short wavelength optical system may include at least one of a scanning laser ophthalmoscope, a fundus camera, and a laser treatment unit. In this case, even when a plurality of types of the OCT light sources having differing center wavelengths are selectively installed, at least one of the scanning laser ophthalmoscope, the fundus camera, and the laser treatment unit can be easily installed without performing a significant amount of adjustment in accordance with the type of the OCT light source.

The short wavelength optical system may include a device (such as an infrared camera or the like) other than the scanning laser ophthalmoscope, the fundus camera, and the laser treatment unit.

The ophthalmic imaging device may further include an anterior ocular segment photographing light source that irradiates the anterior ocular segment photographing light onto the anterior ocular segment of the subject's eye. In this case, the ophthalmic imaging device can capture an image of the anterior ocular segment while illuminating the anterior ocular segment. Thus, it is possible to easily capture the image of the anterior ocular segment.

When the OCT light source is replaced, the anterior ocular segment photographing light source may be replaced in accordance with the OCT light source after replacement. More specifically, when the OCT light source is replaced, the anterior ocular segment photographing light source may be used whose center wavelength is significantly different from the center wavelength of the OCT light source after replacement. For example, when the center wavelength of the OCT light source is 850 nm, the anterior ocular segment photographing light source whose center wavelength is 960 nm may be used, and when the center wavelength of the OCT light source is 1050 nm, the anterior ocular segment photographing light source whose center wavelength is 930 nm may be used. The ophthalmic imaging device may include a wavelength splitting member that is disposed on the first optical path and that divides the first optical path into an optical path of the measuring light and an optical path of the anterior ocular segment photographing light. In this case, the wavelength splitting member that divides the first optical path can accurately divide the first optical path into the optical path of the measuring light and the optical path of the anterior ocular segment photographing light.

The ophthalmic imaging device may include a wavelength splitting member that is disposed on the second optical path and that divides the second optical path into an optical path of the fixation light and an optical path of the short wavelength light.

For example, when at least two OCT light sources having different center wavelengths can be selectively installed in the ophthalmic imaging device, the center wavelength of the anterior ocular segment photographing light source may be between the center wavelengths of the at least two OCT light sources. In this case, in contrast to a case in which the center wavelength of the anterior ocular segment photographing light source is made to be a longer wavelength than the center wavelength of each of the plurality of OCT light sources, there is no need to use an expensive imaging element that can receive light having a long wavelength. When the center wavelength of the anterior ocular segment photographing light source is made to be a shorter wavelength than the center wavelength of each of the plurality of OCT light sources, dividing the common optical path by the wavelength splitting member may be likely to be more difficult. In this case, by making the center wavelength of the anterior ocular segment photographing light source to be between the center wavelengths of the at least two OCT light sources, the optical path can be appropriately divided, and also, an image of the anterior ocular segment can be easily captured. Further, the wavelength splitting member that divides the first optical path may be replaced in accordance with the OCT light source. In this case also, the wavelength splitting member that divides the first optical path can accurately divide the first optical path into the optical path of the measuring light and the optical path of the anterior ocular segment photographing light.

Another aspect of an ophthalmic imaging device exemplified in the present disclosure includes a first optical system and a second optical system. The first optical system includes an optical coherence tomography unit that acquires an OCT signal of a tissue of a subject's eye by dividing OCT light emitted from an OCT light source into measuring light and reference light and receiving interference light of the reference light and reflected light of the measuring light reflected by the tissue. The second optical system irradiates light in a second wavelength region onto the subject's eye. A wavelength of the second wavelength region is shorter than a wavelength of a first wavelength region including a wavelength region of the OCT light. The first wavelength region includes a wavelength of an OCT light source used in SD-OCT and a wavelength of an OCT light source used in SS-OCT. The ophthalmic imaging device further includes a wavelength splitting member. The wavelength splitting member is disposed on a common optical path of light irradiated from the first optical system and light irradiated from the second optical system. The wavelength splitting member divides the common optical path into a first optical path and a second optical path by splitting light that passes through a mirror surface of the wavelength splitting member and light that is reflected by the mirror surface. The first optical path is a path through which light in the first wavelength region passes. The second optical path is a path through which the light in the second wavelength region passes. In this case, the wavelength splitting member can accurately divide the common optical path into the first optical path and the second optical path. Thus, a necessity to perform adjustment along the second path in accordance with the type of the OCT light source can be reduced. Further, it is sufficient that the wavelength splitting member be configured to be capable of dividing the common optical path into the first optical path and the second optical path. Thus, the wavelength splitting member need not necessarily have bandpass characteristics that only allow light of a wavelength of a particular range to pass through or be reflected, for example. As a result, a plurality of types of the OCT light sources having mutually different center wavelengths can easily be selectively installed in the ophthalmic imaging device.

An angle formed between the mirror surface and an optical axis of the common optical path may be more than 45 degrees and less than 90 degrees. In this case, by making the angle formed between the mirror surface of the wavelength splitting member and the optical axis of the common optical path to be more than 45 degrees and less than 90 degrees, the wavelength splitting member is not necessarily required to have high accuracy (mirror thickness, coating, and the like, for example). As a result, a plurality of types of the OCT light sources having mutually different center wavelengths can easily be selectively installed in the ophthalmic imaging device.

In the present disclosure, the angle formed between the mirror surface of the wavelength splitting member that divides the common optical path and the optical axis of the common optical path is more than 45 degrees and less than 90 degrees. However, the angle formed between the mirror surface of the wavelength splitting member that divides the common optical path and the optical axis of the common optical path need not necessarily be more than 45 degrees and less than 90 degrees. The achromatic lens that corrects the chromatic aberration of the measuring light may be included among the one or the plurality of lenses disposed on the optical path of the measuring light. In this case, the ophthalmic imaging device may also be expressed in the following manner. An ophthalmic imaging device comprising: a first optical system that irradiates light whose center wavelength is a first wavelength region, the first wavelength region being an infrared region, the first optical system including an optical coherence tomography unit and an imaging element, the optical coherence tomography unit acquiring an OCT signal of a tissue of a subject's eye by dividing OCT light emitted from an OCT light source into measuring light and reference light and receiving interference light of the reference light and reflected light of the measuring light reflected by the tissue, and the imaging element capturing an image of an anterior ocular segment of the subject's eye by receiving anterior ocular segment photographing light that is infrared light reflected by the anterior ocular segment of the subject's eye; a second optical system that irradiates light whose center wavelength is in a second wavelength region, a wavelength of the second wavelength region being shorter than a wavelength of the first wavelength region, the second optical system including a fixation light source and a short wavelength optical system, the fixation light source projecting a fixation target onto the subject's eye by emitting fixation light that is visible light, the short wavelength optical system performing at least one of capturing an image of an ocular fundus of the subject's eye and irradiating treatment light onto the subject's eye by using short wavelength light, and the short wavelength light having a wavelength that is shorter than a wavelength of the measuring light and shorter than a wavelength of the anterior ocular segment photographing light; and a wavelength splitting member disposed on a common optical path of the measuring light, the anterior ocular segment photographing light, the fixation light, and the short wavelength light, the wavelength splitting member dividing the common optical path into a first optical path and a second optical path, the first optical path being an optical path through which the measuring light and the anterior ocular segment photographing light pass, the second optical path being an optical path through which the fixation light and the short wavelength light pass, an achromatic lens being included among the one or the plurality of lenses disposed on the optical path of the measuring light, and the achromatic lens correcting the chromatic aberration of the measuring light.

### Embodiments

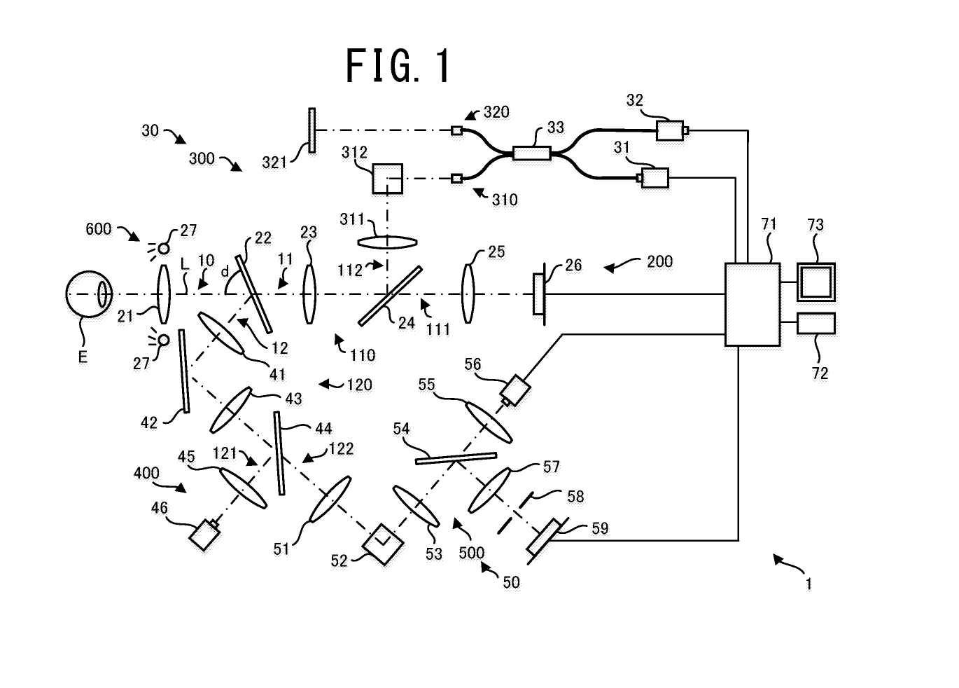

Hereinafter, an exemplary embodiment of the present invention will be explained with reference to the drawings. First, an overall configuration of an ophthalmic imaging device 1 of the present embodiment will be explained with reference to FIG. 1. The ophthalmic imaging device 1 of the present embodiment includes a first optical system 110 and a second optical system 120. The first optical system 110 is an optical system that irradiates light whose center wavelength is in a first wavelength region. The first wavelength region is an infrared region. The second optical system 120 is an optical system that irradiates light whose center wavelength is in a second wavelength region. A wavelength of the second wavelength region is shorter than a wavelength of the first wavelength region. The first optical system 110 includes an anterior ocular segment observation optical system 200 and an OCT optical system 300. The second optical system 120 includes a fixation optical system 400 and a scanning laser ophthalmoscope (SLO) optical system 500. Hereinafter, infrared light received by an imaging element 26 of the anterior ocular segment observation optical system 200 is referred to as anterior ocular segment photographing light. OCT measuring light emitted onto a subject's eye E in order for the OCT optical system 300 to acquire an OCT signal is referred to simply as measuring light. Light emitted from the fixation optical system 400 in order to project a fixation target is referred to as fixation light. Light emitted from the SLO optical system 500 in order to capture an image of the ocular fundus of the subject's eye E is referred to as short wavelength light. The ophthalmic imaging device 1 further includes an alignment marker projecting optical system 600, a control unit 71, a memory 72, and a display 73.

In the ophthalmic imaging device 1, an objective lens 21 and a first wavelength splitting member 22 are disposed on a common optical path 10 of the anterior ocular segment photographing light, the measuring light, the fixation light, and the short wavelength light. The first wavelength splitting member 22 divides the common optical path 10 into a first optical path 11 and a second optical path 12. The first optical path 11 is an optical path through which the measuring light and the anterior ocular segment photographing light pass. The second optical path 12 is an optical path through which the fixation light and the short wavelength light pass. The measuring light and the anterior ocular segment photographing light are infrared light. The fixation light is visible light. The wavelength of the short wavelength light is shorter than the wavelength of the measuring light and shorter than the wavelength of the anterior ocular segment photographing light. At least one of a dichroic mirror, a dichroic prism, and the like may be used as the first wavelength splitting member 22, for example. As an example, the dichroic mirror is used as the first wavelength splitting member 22 of the present embodiment. The first wavelength splitting member 22 of the present embodiment allows the measuring light and the anterior ocular segment photographing light to pass through the first wavelength splitting member 22, and reflects the fixation light and the short wavelength light, thus dividing the common optical path 10. However, the passing light and the reflected light may be reversed.

A second wavelength splitting member 24 is disposed on the first optical path 11. In the present embodiment, a lens 23 is disposed on the optical path from the first wavelength splitting member 22 to the second wavelength splitting member 24. The second wavelength splitting member 24 divides the first optical path 11 into an anterior ocular segment photographing light optical path 111 and a measuring light optical path 112. A dichroic mirror, a dichroic prism, or the like may be used as the second wavelength splitting member 24, for example. Further, for example, another beam splitter (a half mirror, a perforated mirror, a combination of a half mirror and a wavelength filter, or the like) may be used as the second wavelength splitting member 24. The second wavelength splitting member 24 of the present embodiment allows the anterior ocular segment photographing light to pass through the second wavelength splitting member 24, and reflects the measuring light, thus dividing the first optical path 11. However, the second wavelength splitting member 24 may divide the first optical path 11 by reflecting the anterior ocular segment photographing light and allowing the measuring light to pass through the second wavelength splitting member 24.

The anterior ocular segment observation optical system 200 is provided on the anterior ocular segment photographing light optical path 11 that includes the common optical path 10 and the first optical path 11. In the present embodiment, the anterior ocular segment observation optical system 200 is used to obtain a front face observation image of the anterior ocular segment. In the present embodiment, the anterior ocular segment observation optical system 200 includes the objective lens 21, the first wavelength splitting member 22, the lens 23, the second wavelength splitting member 24, a lens 25, and the imaging element 26. The imaging element 26 may be a two-dimensional imaging element, such as a CCD or the like.

In the present embodiment, a plurality of infrared light sources of the alignment marker projecting optical system 600 are used as an anterior ocular segment photographing light source 27 that irradiates the anterior ocular segment photographing light onto the anterior ocular segment of the subject's eye E. The alignment marker projecting optical system 600 is used in order to position optical systems with respect to the subject's eye E. In the present embodiment, the plurality of infrared light sources of the alignment marker projecting optical system 600 are provided on concentric circles centering on a photographing light axis L. An infrared light source may be provided separately from the plurality of infrared light sources of the alignment marker projecting optical system 600, as the anterior ocular segment photographing light source 27. A center wavelength of the anterior ocular segment photographing light source 27 is 930 nm, for example. The imaging element 26 captures an image of the anterior ocular segment of the subject's eye E by receiving the anterior ocular segment photographing light reflected by the anterior ocular segment of the subject's eye E. A signal output from the imaging element 26 is input into the control unit 71. The control unit 71 is connected to the memory 72 and the display 73. On the basis of the input signal, the control unit 71 may generate a front face image of the anterior ocular segment of the subject's eye E and store the generated front face image in the memory 72. The control unit 71 may cause the display 73 to display the generated front face image.

A measurement optical system 310 of the OCT optical system 300 is provided on the measuring light optical path 112 that includes the common optical path 10 and the first optical path 11. The OCT optical system 300 is an optical system of an OCT unit 30. The OCT optical system 300 uses the principle of OCT and is used to acquire an OCT signal of a tissue of the subject's eye E. In the present embodiment, the OCT optical system 300 includes an OCT light source (measuring light source) 31, a detector (photodetection element) 32, a coupler 33, the measurement optical system 310, and a reference optical system 320. The OCT light source 31 emits light (OCT light) that is used to acquire the OCT signal. The coupler 33 divides the light emitted from the OCT light source 31 into measuring light and reference light. The coupler 33 of the present embodiment combines the measuring light reflected by a tissue of the subject's eye E (the ocular fundus, the anterior ocular segment, or the like) and the reference light generated by the reference optical system 320, and causes the combined interference light to be detected by the detector 32.

The measuring light optical system 310 guides the measuring light divided by the coupler 33 to a tissue of the subject's eye E, and returns the measuring light reflected by the tissue of the subject's eye E to the coupler 33. In the present embodiment, the measuring light optical system 310 shares the optical path from the objective lens 21 to the second wavelength splitting member 24 with the anterior ocular segment observation optical system 200. The measuring light optical system 310 further includes a lens 311 and a scanning unit 312. The scanning unit 312 includes an optical scanner and a drive unit. The optical scanner is driven by the drive unit and thus can deflect the measuring light. In the present embodiment, two galvanometer mirrors that can deflect the measuring light in mutually different directions are used as the optical scanner. However, another device that deflects light (at least one of a polygon mirror, a resonant scanner, an acousto-optic device, and the like, for example) may be used as the optical scanner.

The reference optical system 320 generates the reference light and returns the generated reference light to the coupler 33. In the present embodiment, the reference optical system 320 generates the reference light by reflecting the reference light divided by the coupler 33 by using a reference mirror 321. However, the configuration of the reference optical system 320 can be changed. For example, the reference optical system 320 may allow the incident light from the coupler 33 to pass through without reflecting the light, and return the light to the coupler 33.

The detector 32 detects the interference signal of the measuring light and the reference light. In the present embodiment, the principle of Fourier-domain OCT are used. In the Fourier-domain OCT, the spectral intensity (a spectral interference signal) of the interference light is detected by the detector 32, and a complex OCT signal is acquired through a Fourier transform of the spectral intensity data. As examples of the Fourier-domain OCT, spectral-domain OCT (SD-OCT), swept-source OCT (SS-OCT), and the like may be used. For example, time-domain OCT (TD-OCT) and the like may also be used. In the present embodiment, SD-OCT is installed in the ophthalmic imaging device 1. For example, in the ophthalmic imaging device 1, a configuration may be adopted in which SS-OCT can be installed in place of the SD-OCT.

In the case of SD-OCT, for example, a low-coherence light source (a broadband light source) may be used as the OCT light source 31. In the case of the SD-OCT, for example, a spectroscopic optical system (a spectrometer), which diffracts the interference light into individual frequency components (individual wavelength components), may be provided in the vicinity of the detector 32 on the optical path of the interference light. In the case of the SD-OCT, the center wavelength of the OCT light source 31 is 850 nm, for example. In the case of SS-OCT, for example, a wavelength scanning light source (a wavelength tunable light source), which can change the emitted wavelength at a high speed in terms of time, may be used as the OCT light source 31. In this case, the OCT light source 31 may include the light source, a fiber ring resonator, and a wavelength selection filter. Examples of the wavelength selection filter include a filter combining a diffraction grating and a polygon mirror, a filter using a Fabry-Perot etalon structure, and the like. In the case of the SS-OCT, the center wavelength of the OCT light source 31 is 1050 nm, for example.

The OCT signal output from the detector 32 is input into the control unit 71. On the basis of the OCT signal, the control unit 71 may generate data of a tomographic image and the like of a tissue of the subject's eye E and store the generated data in the memory 72. The control unit 71 may cause the display 73 to display the generated tomographic image and the like.

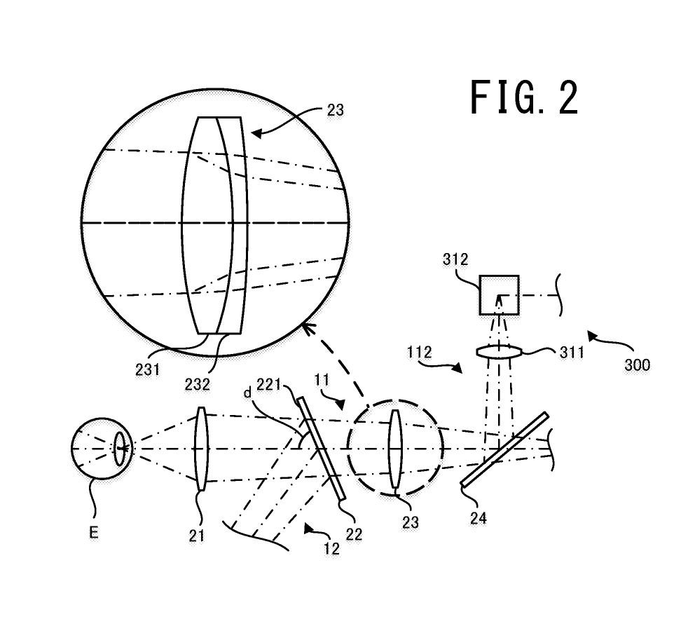

Next, the optical systems disposed on the measuring light optical path 112 of the present embodiment will be explained with reference to FIG. 2. In FIG. 2, only the optical systems disposed from the subject's eye E to the scanning unit 312 are illustrated, and other members are not illustrated. The anterior ocular segment photographing light source 27 is also not illustrated. The lens 23 is also illustrated by being partially enlarged.

As described above, the measuring light and the anterior ocular segment photographing light are infrared light, and the fixation light is visible light. Further, the wavelength of the short wavelength light is shorter than the wavelength of the measuring light and the wavelength of the anterior ocular segment photographing light. Thus, the wavelength of the short wavelength light and the wavelength of the fixation light are shorter than the wavelength of the measuring light and the wavelength of the anterior ocular segment photographing light. It is sufficient that the first wavelength splitting member 22 be capable of dividing the common optical path 10 into the first optical path 11 (the optical path of the measuring light and the anterior ocular segment photographing light) and the second optical path 12 (the optical path of the short wavelength light and the fixation light). Thus, for example, the first wavelength splitting member 22 need not necessarily have band pass characteristics that only allow light of a wavelength of a specific range to pass through or be reflected. Further, an angle d between a mirror face 221 of the first wavelength splitting member 22 and the optical axis of the common optical path 10 is larger than 45 degrees and less than 90 degrees. In general, with light branching characteristics of the dichroic mirror or the dichroic prism, the smaller the optimized incidence angle, the easier it is to impart robustness with respect to the change in the incidence angle. Thus, the first wavelength splitting member 22 is not necessarily required to have high accuracy (the mirror thickness, coating, or the like, for example). As a result, even when the OCT light source 31 whose center wavelength is close to the wavelength of the short wavelength light and the wavelength of the fixation light (the OCT light source 31 whose center wavelength is 850 nm, for example) is used, the first optical path 11 can be appropriately divided. Therefore, a plurality of types of the OCT light sources 31 having mutually different center wavelengths, can easily be selectively installed in the ophthalmic imaging device 1 in a state in which much of the design is shared. For example, as the OCT light source 31, each of the OCT light source whose center wavelength is 850 nm and the OCT light source whose center wavelength is 1050 nm can be selectively installed in the ophthalmic imaging device 1 in a state in which the configuration of the first wavelength splitting member 22 and the like is the same. For example, the first wavelength splitting member 22 can divide the common optical path 10 by allowing light of the first wavelength region that includes both the wavelength of the OCT light source used in the SD-OCT and the wavelength of the OCT light source used in the SS-OCT to pass through, and by reflecting the light of the second wavelength region, a wavelength of which is a shorter than a wavelength of the first wavelength region. As described above, the passing light and the reflected light may be reversed.

Preferably, the angle d is larger than 45 degrees and less than 80 degrees. More preferably, the angle d is more than 45 degrees and less than 70 degrees. When coating the dichroic mirror required to have the high degree of accuracy, such as described above, multiple layers of film are necessary, and it is known that there is a possibility of warping due to that stress (refer to PCT International Application Publication No. WO2015-137183, for example). This impact can be alleviated by making the base material of the dichroic mirror (glass or the like, for example) to be thicker. However, with respect to the optical system that allows the light to pass through the dichroic mirror, when the optical path length that passes through the dichroic mirror is long, the influence of spherical aberration and astigmatism resulting from parallel plates may increase. In the optical system that allows the light to pass through the dichroic mirror, with respect to the thickness of the dichroic mirror, the actual optical path length becomes larger in proportion to the inverse of the cosine of the incidence angle. Thus, by setting the angle d to be the above-described angle and making the incidence angle smaller, even if the dichroic mirror is made thicker, the increase in the optical path length can be suppressed. In this way, the influence of the spherical aberration and the astigmatism can be suppressed while reducing the warping.

Among the lenses (such as the objective lens 21, the lens 23, the lens 311, and the like) disposed on the measuring light optical path 112, an achromatic lens may be included that corrects chromatic aberration of the measuring light. The achromatic lens may include at least one positive lens and at least one negative lens. The achromatic lens may be a cemented lens in which the at least one positive lens and the at least one negative lens are joined together. In the present embodiment, the light that is reflected by the tissue of the subject's eye E and passes through the objective lens 21 and the first wavelength splitting member 22 passes through the lens 23. For example, the lens 23 may be the achromatic lens. More specifically, the lens 23 may be the cemented lens in which a positive lens 231 and a negative lens 232 are joined together. The dispersion of the light (the Abbe number) may be different for the positive lens 231 and the negative lens 232. For example, the positive lens 231 may be a low-dispersion lens and the negative lens 232 may be a high-dispersion lens. For example, Japanese Patent Application Laid-Open No. 2017-184788 discloses a cemented lens that can be used as the achromatic lens, the relevant portions of which are incorporated by reference.

When the achromatic lens is included in the lenses disposed on the measuring light optical path 112, for example, the achromatic lens may be configured to be able to correct both the chromatic aberration of wavelengths in the vicinity of 850 nm (wavelengths from 800 nm to 900 nm, for example) and the chromatic aberration of wavelengths in the vicinity of 1050 nm (wavelengths from 1000 nm to 1100 nm, for example). For example, the achromatic lens may be configured to be able to correct the chromatic aberration of wavelengths from the vicinity of 850 nm to the vicinity of 1050 nm (wavelengths from 800 nm to 1100 nm, for example). In this case, even when the OCT light source 31 is replaced by a light source having a different center wavelength, the influence of chromatic aberration resulting from the difference in the center wavelength of the OCT light source 31 can be suppressed. The ophthalmic imaging device 1 need not necessarily include the achromatic lens.

A coating that suppresses reflection of a plurality of types of OCT light having differing center wavelengths may be applied to at least one of the lenses (such as the objective lens 21, the lens 23, the lens 311, and the like) disposed on the measuring light optical path 112. The coating may suppress both the reflection of light having a wavelength in the vicinity of 850 nm and the reflection of light having a wavelength in the vicinity of 1050 nm. When replacing the OCT light source 31, at least one of the lenses disposed on the measuring light optical path 112 may be replaced with a lens that is appropriate for the wavelength of the OCT light source 31.

As shown in FIG. 1, a third wavelength splitting member 44 is disposed on the second optical path 12. A lens 41, a total reflection mirror 42, and a lens 43 are provided in order on the optical path from the first wavelength splitting member 22 to the third wavelength splitting member 44. The third wavelength splitting member 44 divides the second optical path 12 into a fixation light optical path 121 and a short wavelength light optical path 122. A dichroic mirror, a dichroic prism, or the like may be used as the third wavelength splitting member 44, for example. For example, another beam splitter (such as a half mirror, a perforated mirror, a combination of a half mirror and a wavelength filter, or the like) may be used as the third wavelength splitting member 44. The third wavelength splitting member 44 of the present embodiment reflects the fixation light, and allows the short wavelength light to pass through the third wavelength splitting member 44, thus dividing the second optical path 12. However, the third wavelength splitting member 44 may allow the fixation light to pass through the third wavelength splitting member 44 and reflect the short wavelength light.

The fixation optical system 400 is provided on the fixation light optical path 121 that includes the common optical path 10 and the second optical path 12. The fixation optical system 400 is used to project a fixation target toward the subject's eye E. In the present embodiment, the fixation optical system 400 include the objective lens 21, the first wavelength splitting member 22, the lens 41, the total reflection mirror 42, the lens 43, the third wavelength splitting member 44, a lens 45, and a fixation light source 46. The fixation light source 46 emits the fixation light that is the visible light. The center wavelength of the fixation light source 46 of the present embodiment is 590 nm, for example.

The fixation light emitted from the fixation light source 46 passes through the lens 45, the third wavelength splitting member 44, the lens 43, the total reflection mirror 42, and the lens 41, and is reflected by the first wavelength splitting member 22. Further, the fixation light passes through the objective lens 21 and is concentrated onto the ocular fundus of the subject's eye E. The subject visually recognizes the visible light as the fixation target. In this way, the subject's eye E is fixated.

The SLO optical system 500 is provided on the short wavelength light optical path 122 that includes the common optical path 10 and the second optical path 12. The SLO optical system 500 is an SLO optical system that is a short wavelength optical system 50 of the present embodiment. In the present embodiment, the SLO optical system 500 is used to acquire a front face image of the ocular fundus of the subject's eye E. In the present embodiment, the SLO optical system 500 shares the optical path from the objective lens 21 to the third wavelength splitting member 44 with the fixation optical system 400. The SLO optical system 500 further includes a lens 51, a scanning unit 52, a focusing lens 53, a beam splitter 54, a collimate lens 55, a laser light source 56, a condensing lens 57, a confocal opening 58, and a photodetection element 59.

The SLO optical system 500 irradiates laser light emitted from the laser light source 56 onto the ocular fundus of the subject's eye E. The photodetection element 59 receives the light reflected by the ocular fundus of the subject's eye E. In the present embodiment, the laser light source 56 emits the laser light that is near infrared light, as the short wavelength light. The center wavelength of the laser light source 56 of the present embodiment is 780 nm, for example. An LED light source, an SLD light source, or the like may be used as the laser light source 56, for example. The focusing lens 53 may be capable of adjusting a position of an optical axis direction in accordance with a refraction error of the subject's eye E. In this case, the ophthalmic imaging device 1 may be provided with a drive mechanism that displaces the position of the focusing lens 53.

The scanning unit 52 is disposed in the optical path of the laser light. In the present embodiment, the scanning unit 52 is used to scan the laser light in a lateral direction (an XY direction) on the ocular fundus. In the present embodiment, the scanning unit 52 includes two optical scanners (a resonant scanner and a galvanometer mirror, for example). By driving the two optical scanners, the scanning unit 52 performs a two-dimensional scan, on the ocular fundus of the subject's eye E, of the laser light from the laser light source 56.

In the present embodiment, the condensing lens 57, the confocal opening 58 (a pinhole plate, for example), and the photodetection element 59 are disposed on the reflection side of the beam splitter 54. The confocal opening 58 is disposed in a position conjugated with the ocular fundus of the subject's eye E.

Here, the laser light emitted from the laser light source 56 enters the scanning unit 52, via the collimate lens 55, the beam splitter 54, and the focusing lens 53. Then, a reflection direction of the laser light is changed by the scanning unit 52. The laser light that passes through the scanning unit 52 passes through the lens 51, the third wavelength splitting member 44, and the lens 43 and is then reflected by the total reflection mirror 42. Further, the laser light passes through the lens 41, and is reflected by the first wavelength splitting member 22. Then, the laser light passes through the objective lens 21 and is concentrated on the ocular fundus of the subject's eye E.

The laser light reflected by the ocular fundus of the subject's eye E travels along the optical path from the objective lens 21 to the beam splitter 54. The laser light is reflected by the beam splitter 54, passes through the condensing lens 57 and the confocal opening 58, and is received by the photodetection element 59. A signal output by the photodetection element 59 is input to the control unit 71. On the basis of the input signal, the control unit 71 may generate the front face image of the ocular fundus of the subject's eye E and store the generated image in the memory 72. The control unit 71 may cause the display 73 to display the generated front face image.

Various modifications may be made to the above-described embodiment. For example, in the above-described embodiment, a SLO is used as the short wavelength optical system 50. However, it is sufficient that the short wavelength optical system 50 perform at least one of capturing an image of the ocular fundus of the subject's eye E and irradiating the treatment light onto the subject's eye E by using the short wavelength light whose wavelength is shorter than that of the measuring light and that of the anterior ocular segment photographing light. For example, the ophthalmic imaging device 1 may include at least one of a SLO, a fundus camera, and a laser treatment unit, as the short wavelength optical system 50. For example, Japanese Patent Application Laid-Open No. 2016-013210 discloses a fundus camera, the relevant portions of which are incorporated by reference. For example, Japanese Patent Application Laid-Open No. 2017-153751 discloses a laser treatment unit, the relevant portions of which are incorporated by reference.

When the OCT light source 31 is replaced, the anterior ocular segment photographing light source 27 may be replaced in accordance with the OCT light source 31 after replacement. More specifically, when the OCT light source 31 is replaced, the anterior ocular segment photographing light source 27 may be used whose center wavelength is significantly different from the center wavelength of the OCT light source 31 after replacement. For example, when the center wavelength of the OCT light source 31 is 850 nm, the anterior ocular segment photographing light source 27 whose center wavelength is 960 nm may be used, and when the center wavelength of the OCT light source 31 is 1050 nm, the anterior ocular segment photographing light source 27 whose center wavelength is 930 nm may be used. In this case, the second wavelength splitting member 24 can accurately divide the first optical path 11 into the measuring light optical path 112 and the anterior ocular segment photographing light optical path 111.

For example, when the two OCT light sources 31 having the different center wavelengths can be selectively installed in the ophthalmic imaging device 1, the center wavelength of the anterior ocular segment photographing light source 27 may be between the center wavelengths of the two OCT light sources 31. In this case, the second wavelength splitting member 24 may be replaced in accordance with the OCT light source 31. In this case also, the second wavelength splitting member 24 can accurately divide the first optical path 11 into the measuring light optical path 112 and the anterior ocular segment photographing light optical path 111.

Each of the lenses of the above-described embodiment may be a single lens or may be a lens group formed of a plurality of lenses.

The apparatus and methods described above with reference to the various embodiments are merely examples. It goes without saying that they are not confined to the depicted embodiments. While various features have been described in conjunction with the examples outlined above, various alternatives, modifications, variations, and/or improvements of those features and/or examples may be possible. Accordingly, the examples, as set forth above, are intended to be illustrative. Various changes may be made without departing from the broad spirit and scope of the underlying principles.

What is claimed is:

1. An ophthalmic imaging device comprising:
a first optical system that irradiates light whose center wavelength is a first wavelength region, the first wavelength region being an infrared region, the first optical system including an optical coherence tomography unit and an imaging element, the optical coherence tomography unit acquiring an OCT signal of a tissue of a subject's eye by dividing OCT light emitted from an OCT light source into measuring light and reference light and receiving interference light of the reference light and reflected light of the measuring light reflected by the tissue, and the imaging element capturing an image of an anterior ocular segment of the subject's eye by receiving anterior ocular segment photographing light that is infrared light reflected by the anterior ocular segment of the subject's eye;
a second optical system that irradiates light whose center wavelength is in a second wavelength region, a wavelength of the second wavelength region being shorter than a wavelength of the first wavelength region, the second optical system including a fixation light source and a short wavelength optical system, the fixation light source projecting a fixation target onto the subject's eye by emitting fixation light that is visible light, the short wavelength optical system performing at least one of capturing an image of an ocular fundus of the subject's eye and irradiating treatment light onto the subject's eye by using short wavelength light, and the short wavelength light having a wavelength that is shorter than a wavelength of the measuring light and shorter than a wavelength of the anterior ocular segment photographing light; and
a wavelength splitting member disposed on a common optical path of the measuring light, the anterior ocular segment photographing light, the fixation light, and the short wavelength light, the wavelength splitting member dividing the common optical path into a first optical path and a second optical path by splitting light that passes through a mirror surface of the wavelength splitting member and light that is reflected by the mirror surface, the first optical path being an optical path through which the measuring light and the anterior ocular segment photographing light pass, the second optical path being an optical path through which the fixation light and the short wavelength light pass, and an angle formed between the mirror surface and an optical axis of the common optical path being more than 45 degrees and less than 90 degrees.

2. The ophthalmic imaging device according to claim 1, wherein
an achromatic lens is included among one or a plurality of lenses disposed on an optical path of the measuring light, the achromatic lens correcting chromatic aberration of the measuring light.

3. The ophthalmic imaging device according to claim 1, wherein
a coating is applied to at least one lens among one or a plurality of lenses disposed on an optical path of the measuring light, the coating suppressing reflection of a plurality of types of the OCT light having different center wavelengths.

4. The ophthalmic imaging device according to claim 1, wherein
the short wavelength optical system includes at least one of a scanning laser ophthalmoscope, a fundus camera, and a laser treatment unit.

5. The ophthalmic imaging device according to claim 1, further comprising:
an anterior ocular segment photographing light source that irradiates the anterior ocular segment photographing light onto the anterior ocular segment of the subject's eye.

6. An ophthalmic imaging device comprising:
a first optical system including an optical coherence tomography unit, the optical coherence tomography unit acquiring an OCT signal of a tissue of a subject's eye by dividing OCT light emitted from an OCT light source into measuring light and reference light and receiving interference light of the reference light and reflected light of the measuring light reflected by the tissue;
a second optical system that irradiates light in a second wavelength region onto the subject's eye, a wavelength of the second wavelength region being shorter than a wavelength of a first wavelength region including a wavelength region of the OCT light, and the first wavelength region including a wavelength of an OCT light source used in SD-OCT and a wavelength of an OCT light source used in SS-OCT; and
a wavelength splitting member disposed on a common optical path of light irradiated from the first optical system and light irradiated from the second optical system, the wavelength splitting member dividing the common optical path into a first optical path and a second optical path by splitting light that passes through a mirror surface of the wavelength splitting member and light that is reflected by the mirror surface, the first optical path being a path through which light in the first wavelength region passes, and the second optical path being a path through which the light in the second wavelength region passes.

7. The ophthalmic imaging device according to claim 6, wherein
an angle formed between the mirror surface and an optical axis of the common optical path is more than 45 degrees and less than 90 degrees.
